(19) United States
(12) Patent Application Publication
(10) Pub. No.: US 2019/0151372 A1
(43) Pub. Date: May 23, 2019

(54) PERICYTE CELL EXOSOMES

(71) Applicant: AgeX Therapeutics, Inc., Alameda, CA (US)

(72) Inventors: Dana Larocca, Alameda, CA (US); Paola A. Bignone, Alameda, CA (US); Midori Greenwood-Goodwin, Alameda, CA (US)

(21) Appl. No.: 16/012,487

(22) Filed: Jun. 19, 2018

Related U.S. Application Data
(60) Provisional application No. 62/522,063, filed on Jun. 19, 2017.

(51) Int. Cl. A61K 35/44; C12N 5/0735; C12N 5/071; C12N 5/074; A61P 9/00

(52) U.S. Cl. CPC A61K 35/44; C12N 5/0606; A61P 9/00; C12N 5/0696; C12N 5/0692

(57) ABSTRACT

Compositions and methods of use pertaining to exosomes, and more particularly to exosomes from pericytes and endothelial progenitor cells are presented.

### CROSS-REFERENCE TO RELATED APPLICATIONS

This application claims priority to, and the benefit of, U.S. provisional patent application Ser. No. 62/522,063, filed on Jun. 19, 2017, incorporated herein by reference in its entirety.

### FIELD

The present disclosure relates to exosomes, and more particularly to exosomes from pericyte and endothelial progenitor cells.

### BACKGROUND

Pericytes, also known as Rouget cells or mural cells, are endothelium-associated cells present in small blood vessels. Pericytes play an important role in normal vascular structure and function, including maintenance of the blood-brain barrier, tissue repair and regeneration. As an integral part of vasculature, equivalent to smooth muscle cells for large blood vessels, pericytes wrap around endothelial cells of capillaries, small arterioles and venules, providing a physical barrier and regulating blood flow to the tissue. Pericytes are embedded in basement membrane where they communicate with endothelial cells by means of both direct physical contact and paracrine signaling. Pericytes are also a key component of the neurovascular unit, which includes endothelial cells, astrocytes, and neurons. Additionally, pericytes function in the clearance and phagocytosis of cellular debris and in tissue repair and regeneration. Improper functioning of pericytes can result in abnormal vasculature and contribute to a variety of pathological conditions including ischemic conditions, neurodegenerative disorders, diabetic retinopathy and hepatic fibrosis.

Replacement of pericytes using cell therapy may be useful for treating a number of vascular diseases. Primary pericytes as a source of cells for cell therapy are limited in supply, heterogeneous and have limited scalability. The use of autologous cells for therapy could be limited by the age or health status of the patient. Derivation of pericytes/perivascular stromal cells (PC/PSC) from human embryonic or induced pluripotent stem cells, therefore offers the possibility of a renewable and scalable source of uniform cells for research and development of regenerative therapies.

Exosomes are believed to contain important signaling molecules that may provide the source of trophic factors responsible for some regenerative benefits seen in cell replacement therapy. As such they would provide an alternative to some cell based therapies that would be easier to manufacture on a large scale and potentially safer to administer to a subject in need of cell therapy. Moreover, the risk of immune rejection of the exosomes relative to transplanted cells may also be lower. Accordingly, exosomes may provide an attractive alternative or adjunct to cell based therapies and cell based regenerative medicine.

### SUMMARY

Disclosed herein are, inter alia, methods and compositions for the stimulation and stabilization of vascular tubes and vascular tube networks using nonimmunogenic exosomes.

In certain embodiments, the exosomes are isolated from pericyte-like cells (cells expressing surface markers associated with pericytes and having the functionality of being capable of co-localizing with human umbilical vein endothelial cells (HUVECs) and enhancing tube stability) or pericyte cells.

In other embodiments, the exosomes are isolated from self-renewing perivascular progenitor cells derived from embryonic stem cells.

In another embodiment, the stem cells are human embryonic stem cells (hESC).

In yet another embodiment, the hESCs are from the ESI-017 cell line.

In another embodiment, the pericytes are from PC-M cells.

In another embodiment, the exosomes stabilize tube formation by 73% of total tube length for at least about 1.5 days.

In another embodiment, the exosomes do not illicit an immune response in the subject.

In another embodiment, the exosomes are from the cell line, 30-MV2-6.

In another embodiment, the exosomes display only very low background levels of the MHC I and MHC II antigens.

In other embodiments, the exosomes described herein are administered to a subject for the treatment of a trauma based injury.

In other embodiment, a subject with a vascular disease is treated using the exosomes described herein. In other embodiments, the exosomes are administered to a subject, such that the exosomes come into contact with the subject's vasculature.

In another embodiment, the subject treated with the exosomes described herein is not genetically matched to the exosomes.

### BRIEF DESCRIPTION OF THE DRAWINGS

For a fuller understanding of the nature and advantages of the present invention, reference should be had to the following detailed description taken in connection with the accompanying drawings, in which:

FIG. 1A shows a graph depicting the angiogenic nature of PC-M exosomes. HUVEC tube forming assay results show relative tube length obtained after 12 hours. Values were normalized to tube length obtained when HUVECs were incubated in basal medium containing fetal calf serum. ANOVA analysis of data. Mean+/−SD *p<0.05.

FIG. 1B shows images of PC-M exosomes stabilizing HUVEC vascular tube networks. The majority of the network tube length is preserved (>70%) at 38 h in PC-M treated and serum treated samples. The network formed in the 30-MV2-6 treated sample is unstable showing little or no intact tubes at 38 h.

FIG. 2 shows a graph depicting the lack of MHC Ag on PureStem 30-MV2-6 exosomes. Representative FACs of exosomes captured on CD63 coated beads. 30-MV2-6 exosomes have minimal MHC I or II antigen, similar to negative control HEK293 exosomes. Human dendritic cell exosomes display MHC I and II. All exosomes display transpanin CD81.

### DETAILED DESCRIPTION

Before the compositions and methods of the present disclosure are described, it is to be understood that the invention or inventions disclosed herein are not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. Nothing herein is to be construed as an admission that the invention is not entitled to antedate such disclosure by virtue of prior invention.

Stem cell and cell line derived exosomes have shown promise in animal models as an alternative to stem cells for a wide range of regenerative medicine applications including ischemia, myocardial infarct, stroke, atherosclerosis, and wound healing. However, scale up and production of therapeutic exosomes for clinical use will require scalable, stable and relatively pure production cell lines. Commonly used adult stem cells such as MSCs suffer from poor proliferative capacity, donor variability, population heterogeneity, and phenotypic drift. These limitations present a formidable barrier to translation of early preclinical studies to the clinic.

To address the limitations of cell purity and scalability, in one embodiment, hundreds of clonally pure and highly scalable human embryonic stem cell derived progenitor cell lines were derived (see for example U.S. Patent Application Publication No., US 2010-0184033 incorporated by reference herein in its entirety). In another embodiment, angiogenic exosomes from embryonic progenitor cells demonstrated improved scalability and angiogenic potency compared to adult mesenchymal stem cell (MSC) exosomes. For example, the embryonic endothelial cell line, 30-MV2-6, was expanded to over 75 population doublings (pd) compared to 10-15 pd typical of adult MSCs. Moreover, 30-MV2-6 exosomes had >50-fold higher levels of the angiogenic miR-126 and had 6-fold higher angiogenic potency in a HUVEC tube forming assay than MSCs. Exosome production was stable to at least 50 pd and the potential to scale on a hollow fiber bioreactor was demonstrated. In one embodiment, many distinct cell types including endothelial, smooth muscle, cartilage, bone, fat and pericyte cell lines were identified in our library of over 250 progenitor cell lines. Data indicates the potential of this library to provide a richly diverse source of exosome production lines that can be mined for variety of bio-therapeutic exosomes.

In some embodiments, exosomes isolated from pericyte-like cells can be used to induce the growth and/or stability of vascular tubes. In other embodiments, exosomes isolated from pericyte-like cells described herein enhance vascular tube formation as compared to exosomes isolated from other cell types. In some aspects, exosomes described herein enhance vascular tube formation by at least about 30%, at least about 35%, at least about 40%, at least about 50%, at least about 55%, at least about 60%, at least about 75%, at least about 100%, about 20% to about 150%. In certain embodiments, exosomes described herein, enhance the stimulation of angiogenesis as compared to exosomes isolated from other cell types.

In certain embodiments, exosomes may be used at a concentration of between about 1,000,000 particles/μl to about 10,000,000 particles/μl or at about 3,000,000 to about 4,000,000 particles/μl. Exosomes described herein may be administered to a subject, such that the exosomes come into contact with the subject's vasculature.

In certain embodiments, exosomes described herein stabilize vascular tube networks with between about 20% and 100% of vascular tube network retention after at least about 1.5 days. In other embodiments, between about 50% and 85% of vascular tube networks are retained after 1.5 days. In other embodiments, at least about 73% of vascular tube networks are retained after between about 1 day to about 1 week.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

Stabilization of vascular tube networks using exosomes from embryonic pericyte cell lines. Methods of isolating exosomes from cells have been described, see e.g. US Patent Application Publication No. US2012/0093885. Pericyte-like cell lines derived from human embryonic stem cells (hESC) have previously been described (see for example, U.S. Patent Application Publication No. US2015/0368609 and Greenwood-Goodwin, M., Yang, J., Hassanipour, M., and Larocca, D. (2016) A novel lineage restricted, pericyte-like cell line isolated from human embryonic stem cells. Sci Rep 6, 24403, incorporated herein by reference in their entirety). These cell lines uniformly display pericyte markers CD146, CD105, CD73 but express only minimal levels of stemness markers (CD133, CD144) or the endothelial marker, CD31. Co-culture of the cell line, PC-M, with human umbilical cord endothelial cells (HUVEC) on Matrigel resulted in formation of vascular tubular networks. The tubular networks formed by co-culture with PC-M and HUVEC were stable for up to 6 days whereas the networks formed in the absence of PC-M cells began to disintegrate after 1 day (Id.).

In this example, the secreted exosomes from example cell lines, PC-M, 30-MV2-6 (an endothelial cell line), and MSC-WJ (mesenchymal stem cells from Wharton's jelly) were analyzed to demonstrate their ability to stimulate angiogenesis using the HUVEC vascular tube forming assay. The exosomes were tested in the HUVEC tube forming assay by incubating the exosomes at a dose of about 200×10⁶ particles/50 μl with HUVECs seeded on low growth factor Matrigel in u-well slides. PBS was used as a negative control and medium plus serum was used as a positive control.

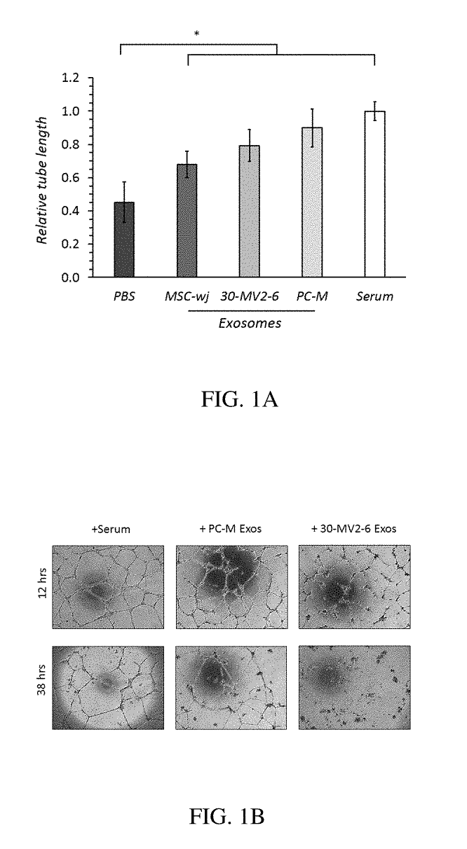

Exosomes derived from MSC-WJ (from Wharton's jelly), 30-MV2-6 (embryonic endothelial cells), and PC-M were found to stimulate angiogenesis (FIG. 1A) with PC-M derived exosomes having higher activity than 30-MV2-6 derived exosomes. MSC-WJ exosomes demonstrated the lowest angiogenic activity.

In addition, incubation of HUVECs with PC-M exosomes was analyzed to determine whether incubation would result in stabilization of HUVEC tube forming networks.

Representative images of triplicate samples are shown for HUVEC vascular tube formation at 12 h and 38 h after exosome addition (FIG. 1B). Network formation of HUVECs at 12 h in basal medium containing PC-M or 30-MV2-6 cell line derived exosomes was equivalent to network formation in complete medium (positive control, which includes serum). The vascular tube network that initially formed when PC-M exosomes or complete medium were added was stabilized at 38 hours, retaining 73% to 83% of total tube length respectively. In contrast, the network formed following 30-MV2-6 exosome treatment was degraded by 38 h. These results indicate that exosomes from pericyte-like cells provide a stabilizing effect on vasculature.

### Example 2

Lack of MHC antigens on embryonic endothelial progenitor derived exosomes. The lower complexity of secreted exosomes demonstrates that they may be less immunogenic than cells. In this example, the expression of MHC class I and II antigens on the surface of exosomes derived from an example PURESTEM® embryonic endothelial cell line, 30-MV2-6, was analyzed to assess their potential immunogenicity. (See, for example, West et al., 2008, Regenerative Medicine vol. 3(3) pp. 287-308, incorporated herein by reference, including supplemental information; and U.S. patent application publication No. US 2010-0184033 filed on Jul. 16, 2009 and titled “Methods to Accelerate the Isolation of Novel Cell Strains from Pluripotent Stem Cells and Cells Obtained Thereby,” and U.S. patent application publication No. US 2016-0108368 both of which are incorporated herein by reference in their entirety.)

Purified exosomes were incubated with magnetic beads conjugated to an anti-CD63 antibody to capture the exosomes that were then incubated with fluorescently tagged antibodies against either MHC I, MHC II, or CD81 antigen. The beads were washed and analyzed by flow cytometry to determine the percentage of beads bearing antigen displaying exosomes. The 30-MV2-6 exosomes were compared to dendritic cell exosomes which are expected to display both MHC I and II, and to HEK293 exosomes which do not display any MHC antigens. All exosomes were expected to display the CD81 antigen.

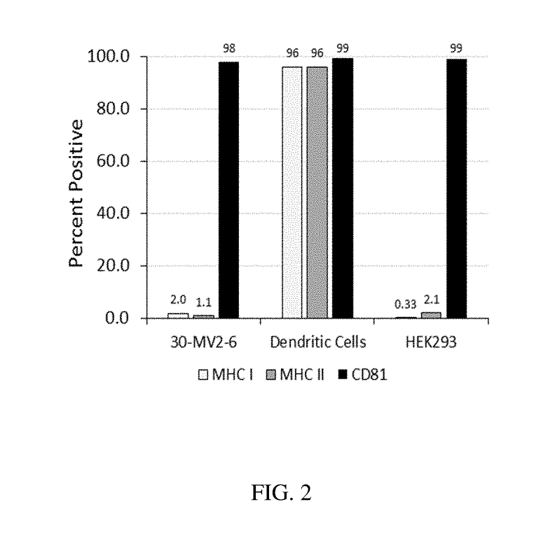

The 30-MV2-6 exosomes displayed only very low background levels of MHC I and MHC II antigens, which was similar to the negative control HEK293 exosomes (FIG. 2). However, dendritic cell exosomes uniformly displayed both MHC antigens (FIG. 2). All exosomes displayed the CD81 antigen. These data indicate a low potential of endothelial cell exosomes to illicit an immune response.

What is claimed is:

1. A composition comprising, exosomes isolated from a pericyte-like cell line, wherein the pericyte-like cell line is derived from pluripotent stem cells and wherein the exosomes are capable of one or both of stimulating or stabilizing the formation of vascular tube networks.

2. The composition of claim 1, wherein the exosomes are nonimmunogenic.

3. The composition of claim 2, wherein the exosomes display only background levels of one or more of MHC I and MHC II antigens.

4. The composition of claim 1, wherein the exosomes are isolated from self-renewing perivascular progenitor cells derived from embryonic stem cells.

5. The composition of claim 1, wherein the stem cells are human embryonic stem cells (hESC) or induced pluripotent stem cells.

6. The composition of claim 1, wherein when the exosomes are capable of retaining vascular tube networks by between about 20% to about 100%.

7. The composition of claim 1, wherein when the exosomes are capable of retaining vascular tube networks by at least about 73%.

8. The composition of claim 1, wherein the exosomes are administered to a subject such that the exosomes come into contact with the subject's vasculature.

9. The composition of claim 8, wherein the exosomes are at a concentration of between about 1,000,000 particles/μl to about 10,000,000 particles/μl.

10. The composition of claim 1, further comprising exosomes isolated from endothelial cell lines.

11. The composition of claim 1, wherein the exosomes enhance vascular tube formation by at least about 30%, at least about at least about 35%, at least about 40%, at least about 50%, at least about 55%, at least about 60%, at least about 75%, at least about 100%, about 20% to about 150%.

12. The composition of claim 11, wherein the enhancement of vascular tube formation is in comparison to exosomes isolated from other cell types.

13. The composition of claim 1, wherein the exosomes express one or more of the markers CD146, CD105, and CD73 but only minimal levels of the markers CD133, CD144, and CD31.

14. The composition of claim 1, wherein the exosomes are capable of stabilizing the formation of vascular tube networks for at least about 1 week.

15. A method for treating a vascular disease, disorder, or traumatic injury in a subject comprising, administering to the subject a composition comprising exosomes isolated from a pericyte-like cell line, wherein the pericyte-like cell line is derived from pluripotent stem cells and wherein the exosomes are capable of one or both of stimulating or stabilizing the formation of vascular tube networks.

16. The method of claim 15, wherein the exosomes are administered to the subject such that they come into contact with the subject's vasculature.

17. The method of claim 15, wherein the exosomes enhance vascular tube formation by at least about 30%, at least about at least about 35%, at least about 40%, at least about 50%, at least about 55%, at least about 60%, at least about 75%, at least about 100%, about 20% to about 150%.

18. The method of claim 17, wherein the enhancement of vascular tube formation is in comparison to exosomes isolated from other cell types.

19. The method of claim 15, wherein the exosomes are at a concentration of between about 1,000,000 particles/μl to about 10,000,000 particles/μl.

20. The composition of claim 15, wherein the exosomes are nonimmunogenic.

21. The composition of claim 20, wherein the exosomes display only background levels of one or more of MHC I and MHC II antigens.

22. The composition of claim 15, wherein the exosomes are isolated from self-renewing perivascular progenitor cells derived from embryonic stem cells.

23. The composition of claim 15, wherein the stem cells are human embryonic stem cells (hESC) or induced pluripotent stem cells.

24. The composition of claim 15, wherein when the exosomes are capable of retaining vascular tube networks by between about 20% to about 100%.

25. The composition of claim 15, wherein when the exosomes are capable of retaining vascular tube networks by at least about 73%.
